(19) United States
(12) Patent Application Publication
(10) Pub. No.: US 2004/0158191 A1
(43) Pub. Date: Aug. 12, 2004

(54) Method and apparatus for treating acute myocardial infarction with hypothermic perfusion

(76) Inventors: Wilfred J. Samson, Saratoga, CA (US); Hoa Nguyen, San Jose, CA (US); Huu Nguyen, San Jose, CA (US); Brady Esch, San Jose, CA (US); Janine Robinson, Half Moon Bay, CA (US)

Correspondence Address: Gunther O. Hanke, Fulwider Patton Lee & Utecht, LLP, Suite 1550, 200 Oceangate, Long Beach, CA, 90802, US

(21) Appl. No.: 10/771,804

(22) Filed: Feb. 4, 2004

Related U.S. Application Data
(63) Continuation-in-part of application No. 10/102,124, filed on Mar. 19, 2002.

(51) Int. Cl. A61M 37/00; H05B 1/00; A61F 7/12

(52) U.S. Cl. 604/6.13; 604/113

(57) ABSTRACT

An apparatus and method are described for efficiently cooling the myocardium while minimizing blood dilution as well as volume buildup within the patient. A flow of cooled fluid is conducted through a percutaneously introduced catheter into the aorta where only a portion thereof is discharged while the remainder is withdrawn from the patient. The much greater flow rate through the catheter that can thereby be maintained without adverse physiological effect serves to minimize the heat gained by the fluid as a result of the catheter's immersion in blood at body temperature. By arranging the catheter such that the return flow surrounds and thereby insulates the supply flow, even colder fluid can be delivered to the myocardium.

### CROSS REFERENCE TO OTHER APPLICATIONS

This application is a continuation-in-part of U.S. patent application Ser. No. 10/102,124, filed on Mar. 19, 2002 which claims the benefit of U.S. patent application Ser. No. 09/384,467, filed on Aug. 27, 1999, which claims the benefit of U.S. provisional application serial No. 60/098,727, filed on Sep. 1, 1998, the specifications of which are hereby incorporated in their entirety.

### FIELD OF THE INVENTION

The present invention relates generally to methods and devices for treatment of heart disease. More particularly, it relates to methods and devices for treating acute myocardial infarction with hypothermic perfusion.

### BACKGROUND OF THE INVENTION

Heart disease is the most common cause of death in the United States and in most countries of the western world. Coronary artery disease accounts for a large proportion of the deaths due to heart disease. Coronary artery disease is a form of atherosclerosis in which lipids, cholesterol and other materials deposit in the arterial walls gradually narrowing the arterial lumen, thereby depriving the myocardial tissue downstream from the narrowing of blood flow that supplies oxygen and other critical nutrients and electrolytes. These conditions can be further exacerbated by a blockage due to thrombosis, embolization or arterial dissection at the site of the stenosis. A severe reduction or blockage of blood flow can lead to ischemia, myocardial infarction and necrosis of the myocardial tissue.

Recent research has indicated that, during the acute stages of myocardial infarction, as much as half of the myocardial tissue at risk can be salvaged by hypothermic treatment of the ischemic area. It is theorized that hypothermia halts the progression of apoptosis or programmed cell death, which causes as much tissue necrosis as the ischemia that precipitated the myocardial infarction. To date, most attempts at hypothermic treatment for acute myocardial infarction have involved global hypothermia of the patient's body, for example using a blood heat exchanger inserted into the patient's vena cava. While this method has shown some efficacy in trials, it has a number of drawbacks. In particular, the need to cool the patient's entire body with the heat exchanger slows the process and delays the therapeutic effects of hypothermia. The more quickly the patient's heart can be cooled, the more myocardial tissue can be successfully salvaged. Global hypothermia has another disadvantage in that it can trigger shivering in the patient. A number of strategies have been devised to stop the patient from shivering, but these add to the complexity of the procedure and have additional risks associated with them. Sequelae due to global hypothermia can be avoided altogether by induction of localized hypothermia of the heart or of the affected myocardium. Localized hypothermia has the additional advantage that it can be achieved quickly because of the lower thermal mass of the heart compared to the patient's entire body. Rapid induction of therapeutic hypothermia gives the best chance of achieving the most myocardial salvage and therefore a better chance of a satisfactory recovery of the patient after acute myocardial infarction.

While blood can be cooled outside the body, the relative fragility of blood cells severely limits the manner and rate at which blood can be pumped and routed. Such restrictions may therefore ultimately limit how quickly therapeutic hypothermia can be induced with the use of blood as the cooling fluid. While such shortcomings are avoided with the use of a non-blood fluid as an injected coolant, other considerations apply such as the build-up of excessive fluid volume within the patient and the dilution of oxygenated blood within the vascular bed.

What would be desirable, but heretofore unavailable, is an apparatus and method for rapid induction of localized therapeutic hypothermia of the heart or of the affected myocardium in a patient experiencing acute myocardial infarction without excessive dilution of the blood nor build-up of fluid volume.

### SUMMARY OF THE INVENTION

In keeping with the foregoing discussion, the present invention provides an apparatus and method for the induction of localized therapeutic hypothermia of the heart by the routing of cooled, physiologically-acceptable fluid at a high flow rate directly to the coronary vasculature while minimizing the introduction of excessive volume and the dilution of oxygenated blood. The apparatus and method provide rapid cooling of the affected myocardium to achieve optimal myocardial salvage in a patient experiencing acute myocardial infarction. The apparatus and method may be used concomitantly with interventional devices so as to effect a cooling of myocardium during interventional procedure.

The apparatus takes the form of a therapeutic hypothermia system including at least one coronary perfusion catheter and a fluid source for delivering a hypothermically-cooled, physiologically-acceptable fluid. The coronary perfusion catheter has an elongated catheter shaft configured for transluminal introduction via an arterial insertion site, such as a femoral, subclavian or brachial artery. The catheter includes a supply lumen and return lumen that each extend from the proximal end of the catheter to its distal end. Fittings at the proximal ends of the lumens allow for the interconnection of the catheter to the appropriate pumping and cooling devices.

The catheter is configured such that the flow of fluid from the supply lumen is divided near the distal end of the catheter between the return lumen through which fluid is conducted back out of the patient and distal ports through which fluid issues from the catheter into the coronary vasculature. Any of various valve configurations may be employed to control the relative flowrates along the two flowpaths. Preferably, such valve is actuatable from the proximal end of the catheter so as to allow for the adjustment of the flows while the catheter is in place within the patient. Appropriate valve configurations may be incorporated in the catheter near its distal or proximal end. Fluid is pumped through the catheter either by pressurizing the fluid in the supply lumen or by pressurizing the supply lumen in combination with subjecting the return lumen to negative pressure. The lumens may be disposed within the catheter in a coaxial or side-by-side arrangement. The catheter may be of the rapid exchange type so as to accommodate a guidewire through its distal end or have an additional full-length lumen to accommodate a guidewire. Additionally, one or more temperature sensors may be embedded in the catheter so as to allow the temperature of the cooling fluid within the catheter or the blood external to the catheter to be monitored. The catheter may also be configured with an occlusion device to constrain the cooling fluid emitted therefrom to the coronary vasculature, especially when a non-blood, oxygen-carrying cooling fluid is employed. The catheter may incorporate one or more thermal insulation layers in its construction. Additionally, the catheter may be configured so as to also perform angioplasty or stent delivery functions.

The catheter configuration of the present invention allows cooling fluid to be delivered to the vasculature targeted for hypothermic treatment at substantially lower temperatures than would otherwise be possible. Because only a portion of the cooling fluid volume flowing through the catheter is actually dispensed from the catheter into the coronary vasculature, substantial flow rates through the supply lumen can be achieved without adversely affecting the vascular bed by excessive dilution of the oxygenated blood or by the introduction of an excessive volume of fluid thereinto. The high flow rate minimizes the temperature gained due to heat transfer from the surrounding blood, which is at body temperature, and in which a substantial length of the deployed catheter is immersed. A high flow rate avails a large volume of fluid for heat adsorption while a high flow velocity also reduces the fluid's exposure time to the elevated temperature. A configuration in which the return lumen surrounds the supply lumen provides a further benefit wherein the return flow insulates the supply flow.

Any of various physiologically-acceptable fluids may be employed. Delivery temperatures of down to 0° C. and delivery rates of up to 100 mL/min can be accommodated in the coronary vasculature. The catheter's distal port may additionally be isolated from the aortic root so as to prevent dispersal of the cooling fluid into the general blood circulation to thereby maximize the rate of cooling of the myocardium. Various occlusion devices or flow control devices can be adapted to or combined with the catheter of the present invention to achieve the desired segmentation. Alternatively, the system of the present invention may be adapted so as to provide for the localized hypothermia of other organs of the body such as the kidneys or lungs.

Cooling fluid supplied from a reservoir is cooled by any of various heat exchange systems preferably just prior to its introduction into the proximal end of the supply lumen. Cooling fluid issuing from the proximal end of the return lumen is preferably recirculated into the supply side upstream of the heat exchanger.

These and other features and advantages of the present invention will become apparent from the detailed description of preferred embodiments which, taken in conjunction with the accompanying drawings, illustrate by way of example principles of the present invention.

### BRIEF DESCRIPTION OF THE DRAWINGS

FIG. 1 is a schematic representation showing the distal end of the catheter of the present invention in place within a patient's heart and the proximal end of the catheter interconnected to a cooling fluid supply system;

FIG. 2 is an enlarged partial cross-sectional view of the distal end of a preferred embodiment catheter of the present invention;

FIG. 3 is an enlarged partial cross-sectional view of the distal end of another preferred embodiment catheter of the present invention;

FIG. 4 is an enlarged partial cross-sectional view of the distal end of another preferred embodiment catheter of the present invention;

FIG. 5 is an enlarged partial cross-sectional view of the distal end of another preferred embodiment catheter of the present invention;

FIG. 6 is an enlarged partial cross-sectional view of the distal end of another preferred embodiment catheter of the present invention;

FIG. 6A is a cross-sectional view taken along lines 6A-6A of FIG. 6;

FIG. 7 is an enlarged partial cross-sectional view of the distal end of another preferred embodiment catheter of the present invention;

FIG. 8 is an enlarged partial cross-sectional view of the distal end of another preferred embodiment catheter of the present invention;

FIG. 9 is an enlarged partial cross-sectional view of the distal end of another preferred embodiment catheter of the present invention;

FIG. 10 is an enlarged partial cross-sectional view of the distal end of another preferred embodiment catheter of the present invention;

FIG. 11 is an enlarged partial cross-sectional view of the distal end of another preferred embodiment catheter of the present invention;

FIG. 12 is an enlarged partial cross-sectional view of the distal end of another preferred embodiment catheter of the present invention;

FIG. 13 is an enlarged partial cross-sectional view of the distal end of another preferred embodiment catheter of the present invention;

FIG. 13A is a cross-sectional view taken along lines 13A-13A of FIG. 13; and

FIG. 14 is an alternative cross-sectional view of the catheter of FIG. 13.

### DETAILED DESCRIPTION OF PREFERRED EMBODIMENTS

The present invention provides an apparatus and method for the induction of therapeutic hypothermia of the heart by hypothermic perfusion of the myocardium through the patient's coronary arteries. The apparatus and method provide for very rapid cooling of the affected myocardium to achieve optimal myocardial salvage in a patient experiencing acute myocardial infarction.

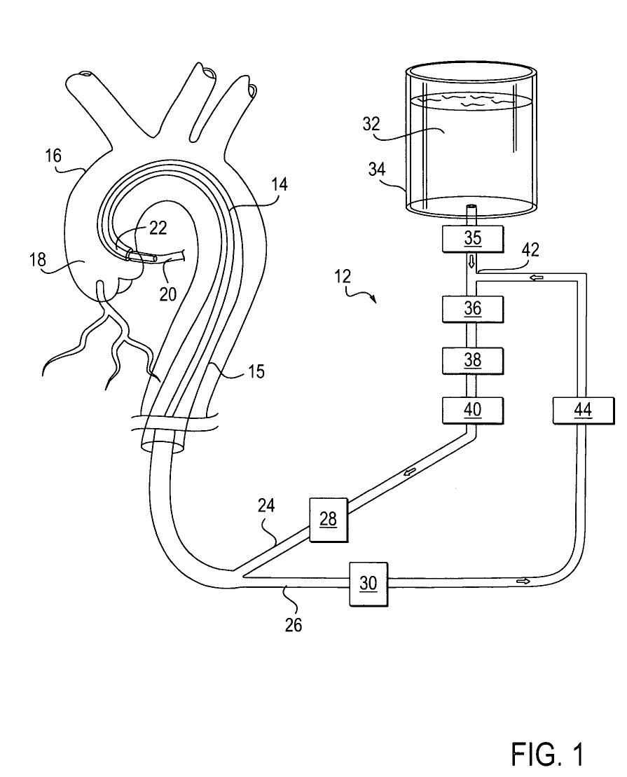

The apparatus takes the form of a therapeutic hypothermia system including at least one coronary perfusion catheter and a fluid source for delivering a hypothermically-cooled, physiologically-acceptable fluid. FIG. 1 is a schematic representation showing the system 12 in its deployed state. A catheter 14 is percutaneously introduced at an arterial insertion site, such as a femoral, subclavian or brachial artery, and advanced through a guiding catheter 15 to and through the aortic arch 16, aortic root 18 and into the right or the left coronary artery 20. A distal port 22 is disposed at or near the catheter's distal end while the proximal ends of a supply lumen 24 and return lumen 26 emerge from near the catheter's proximal end each fitted with an appropriate coupling 28, 30 for interconnection to fluid handling conduits. Fluid 32 from a supply reservoir 34 is routed through a flow meter 35, a pump 36 which forces the fluid through a heat exchanger 38 and a filtration and debubbling device 40 and into the supply lumen 24 of catheter 14. Fluid flowing through return line 26 is recirculated back into the fluid handling system at junction 42. An additional pump 44 may optionally be used to actively draw fluid out through return lumen 26.

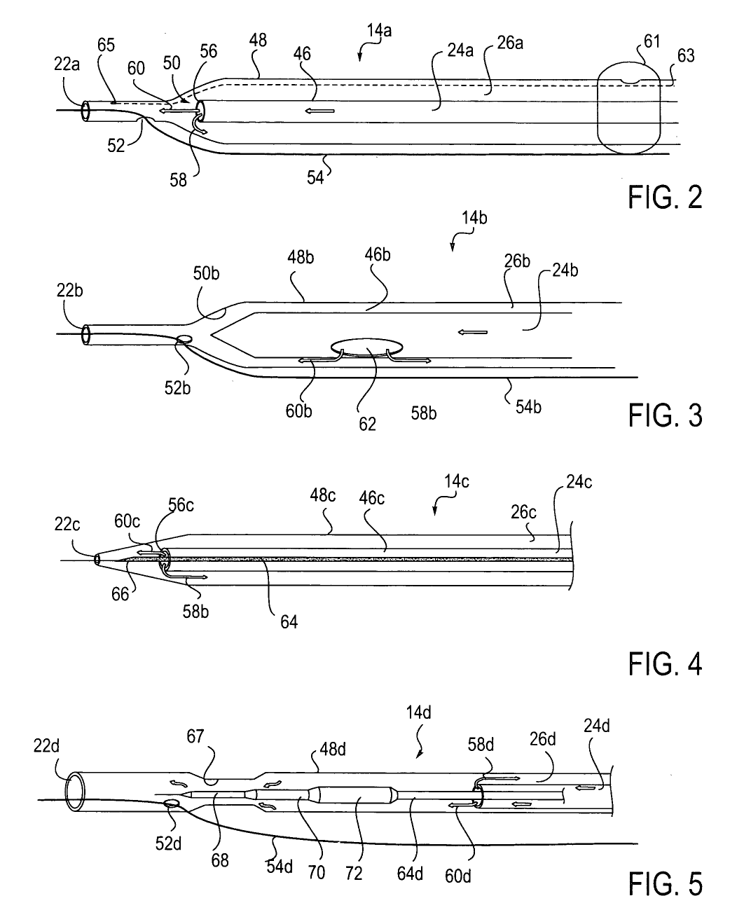

FIG. 2 is an enlarged partial cross-sectional view of the distal end of a preferred embodiment of the catheter of the present invention. The catheter 14a includes an inner tubular member 46 that defines supply lumen 24a and an outer tubular member 48 that defines return lumen 26a. The distal outer tubular member has a tapered inner diameter 50 near its distal end that is proximal to the distal port 22a. A side port 52 may be formed in the side of the distal section of the catheter to accommodate a guide wire 54 to provide for rapid exchange capability as is well known in the art. The inner tubular member 46 is longitudinally shiftable relative to the outer tubular member 48 such that the distal end 56 of inner tubular member 46 can interact with the taper 50 to control the flow of fluid thereby. By shifting the inner tubular member 46 distally, the flow 58 of fluid from the supply lumen 24a back into the return lumen 26a is decreased, while the flow 60 out of the distal port 22a is increased. Shifting the inner tubular member 46 proximally has the opposite effect on the flow distribution. This particular embodiment also illustrates an optional occlusion element 61 that may be fitted to any of the various embodiments described herein. An inflation lumen 63 extends within or along the catheter to its proximal end through which the occlusion member can be inflated and deflated. Additional optional features include an embedded temperature sensor 65 by which the temperature of the cooling fluid issuing from the catheter can be monitored. An additional or alternative temperature sensor can be embedded in the exterior of the catheter near its distal end to gage the temperature of the surrounding blood.

FIGS. 3-6 illustrate various preferred embodiments of a needle-valve type configuration for use in the catheter of the present invention. FIGS. 7-14 illustrate slide-valve type valve configurations for the catheter of the present invention.

FIG. 3 is an alternative embodiment in which the distal end of inner tubular member 46b is sealed, while a side port 62 is formed near the distal end of the inner tubular member. Longitudinally shifting the inner tubular member 46b causes its distal end to interact with the tapered inner surface 50b of the outer tubular member to control the flow thereby. Shifting the inner tubular member distally will reduce flow 60b out the distal port 22b, while increasing return flow 58b.

FIG. 4 illustrates yet another preferred embodiment of the catheter 14c of the present invention. The inner tubular member 46c and outer tubular member 48c are longitudinally fixed relative to one another, while a needle element 64 is longitudinally shiftable along the central axis of the catheter device. The element has a tapered region 66 near its distal end. Longitudinally shifting the needle element will cause the tapered surface to cooperate with the distal port 22c to control flow 60c thereby. Distally shifting the needle element will reduce flow 60c while increasing flow 58c.

FIG. 5 illustrates yet another preferred embodiment of the catheter 14d of the present invention. The outer tubular member 48d has a section of reduced inner diameter 67 proximal to distal port 22d. Needle element 64d has near its distal end a series of discreetly stepped outer diameters 68, 70 and 72. Longitudinally shifting the needle so as to place a pre-selected one of said stepped sections adjacent the section of reduced inner diameter 67 will control the flow 60d thereby. Distally shifting the needle element will cause flow 60d to decrease while increasing flow 58d. Alternatively, a greater or lesser number of sections of discretely stepped diameters may be employed to facilitate the regulation of flow.

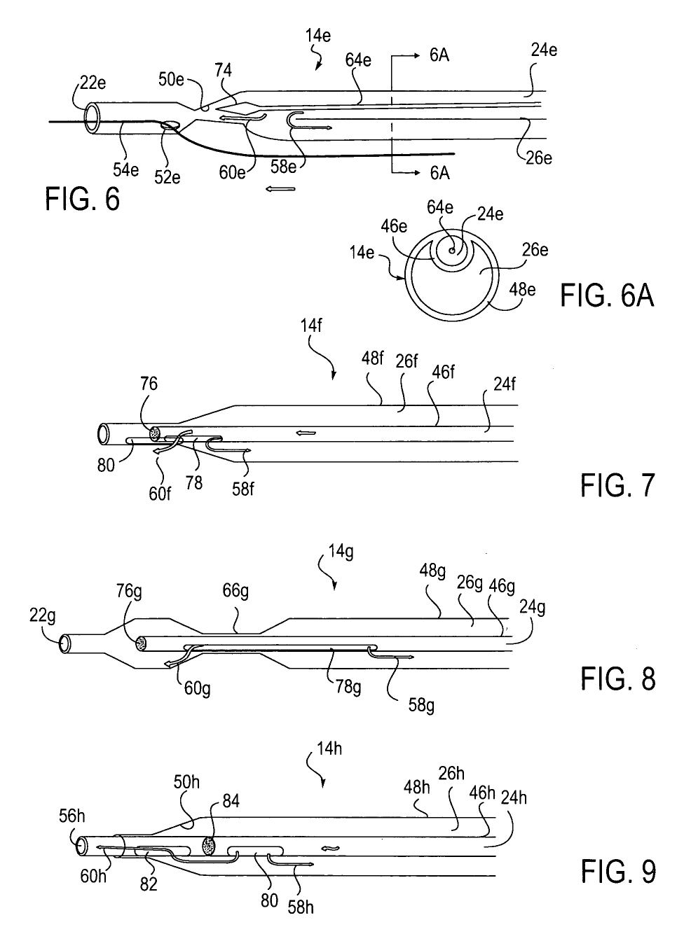

FIG. 6 is yet another preferred embodiment of the catheter 14e of the present invention. Outer tubular member 48e and inner tubular member 46e are arranged in an offset orientation as is visible in the cross-sectional view shown in FIG. 6a. In the embodiment shown, needle element 64e extends through the supply lumen 24e and enlarged conical distal end 74 is configured to interact with the tapered inner diameter 50e of the catheter 14e. Distally shifting the needle element 64e will reduce flow 60e past the tapered section 50e and out distal port 22e, while increasing return flow 58e. A proximal shift of the needle element will have the opposite effect.

FIG. 7 is a preferred embodiment of a slide-valve catheter valve configuration. The catheter 14f includes an inner tubular member 46f that defines supply lumen 24f which is surrounded by an outer tubular member 48f which defines return lumen 26f therebetween. The inner tubular member is sealed 76 at its distal end and has a side port 78 proximate thereto, while the outer tubular member has a side port 80 situated near its distal end and is sealed 76 at its distal end. By longitudinally shifting the inner tubular member relative to the outer tubular member, the overlap of the two lumen side ports can be adjusted so as to control the flow thereby. Distally shifting the inner tubular member from the position shown in FIG. 7 will increase the flow 60f while decreasing the flow 58f. A proximal shift will have the opposite effect.

FIG. 8 illustrates another alternative embodiment, wherein inner tubular member 46g is sealed 76g at its distal end and includes a longitudinal slot 78g formed along its side. The outer tubular member 48g includes a section of reduced inner diameter 66g. Longitudinal shifting of the inner tubular member relative to the outer tubular member allows the flow 60g issuing from the catheter through port 22g and the return flow 58g to be adjusted.

FIG. 9 is an illustration of another preferred embodiment of catheter 14h of the present invention. Inner tubular member 46h again defines inner lumen 24h, while outer tubular member 48h defines return lumen 26h therebetween. The inner tubular member includes two side ports 80 and 82 separated by a divider element 84. Longitudinally shifting inner tubular member 46h relative to outer tubular member 48h causes the distal side port 82 to be shifted relative to the tapered inner diameter 50h of outer tubular member. Distally shifting the inner tubular member will reduce the area of distal side port 82 exposed to the flow of cooling fluid to reduce the flow 60h out the distal end 56h of the inner tubular member. A proximal shift of the inner tubular member from the position illustrated will increase flow 60h, while decreasing return flow 58h.

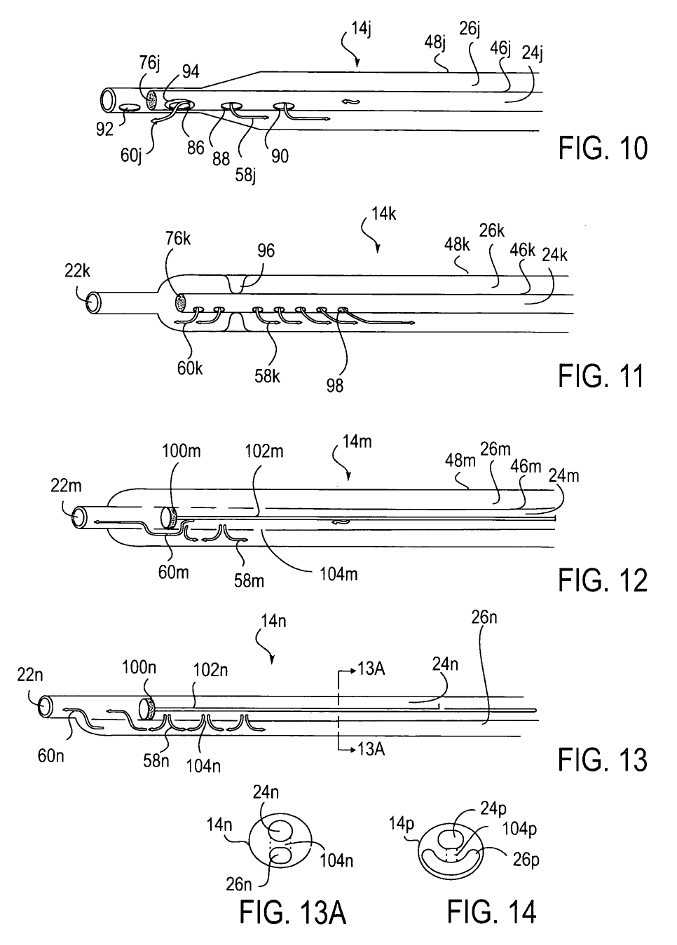

FIG. 10 illustrates a preferred embodiment of catheter 14j. The tubular member 46j has a series of side ports 86, 88 and 90 formed therein, while outer tubular member 48j has side ports 92-94 formed therein. The tubular member is sealed 76j at its distal end. By longitudinally shifting the tubular member distally, more of the side ports of the inner tubular member become aligned with the side ports of the outer tubular member to thereby increase the flow 60j out of the catheter, while decreasing return flow 58j. This embodiment is not limited to the number of ports illustrated in FIG. 10, additional or fewer ports can be formed both in the inner tubular member and/or the outer tubular member.

FIG. 11 is another preferred embodiment of the present invention. Outer tubular member 48k has a section of reduced inner diameter 96 formed therein. The inner tubular member 46k has a series of side ports 98 formed therein. By longitudinally shifting the inner tubular member, the number of side ports on either side of the restriction can be adjusted so as to control the flow 60k from the catheter relative to the return flow 58k.

In FIG. 12, inner tubular member 46m is situated within outer tubular member 48m to define respectively supply lumen 24m and annular return lumen 26m. A plunger element 100m is longitudinally positioned within inner tubular member and is longitudinally shiftable by manipulation of control wire 102m. Side ports 104m formed within the inner tubular member allow the flow of fluid into and out of the lumen within inner tubular member 46m. By shifting the plunger element 100m proximally via manipulation of control wire 102m, an increasing number of the side ports become available for the influx of fluid from the outer tubular members to thereby increase flow 60m out the distal port 22m. Distally shifting the plunger element will have the opposite effect to increase the return flow 58m.

FIG. 13 illustrates another preferred embodiment of the present invention. The catheter 14n includes supply lumen 24n and return lumen 26n arranged in a side-by-side configuration as is shown across sectional view in FIG. 13a. A series of ports 104n set the two lumens into fluid communication with one another, while a plunger element 100n is longitudinally shiftable within supply lumen by manipulation of control wire 102n. Distally shifting the plunger will have the effect of decreasing distal flow 60n for the flow out to support 22n, while a proximal shift will have the opposite effect.

FIG. 14 illustrates an alternative cross-sectional configuration of the catheter shown in FIG. 13 in which the return lumen 26p has a non-circular cross-section as illustrated.

The various dimensions and relative orientations of the various components of the above-described preferred embodiments can be selected to enable access to a targeted vascular bed and to provide the desired flow rates of cooling fluid. Access to a coronary artery would be facilitated by a catheter size of from 3-5 French with a total length of approximately 135-145 centimeters. The typical materials for construction of the shaft may include polyetheylene, polyimide, polyamide, polyurethane, stainless steel or nitonol hypo-tubing, polyamide/polyether blends (e.g., PeBax) and stainless steel wire reinforcement. The catheter may include a lubricious coating (e.g., silicon or hydro-gel) and the proximal end of the catheter may be composed of polycarbonate, acrylic, rigid PVC, or similar, with sealing inserts such as silicon, viton, neoprene, or Teflon. Cooling fluid for the catheter could be sourced from a standard IV saline bag and pumped via means of an external high pressure pump. Prior to delivery into the catheter, the fluid could be pumped through a heat exchanger which could be a thermoelectric cooler, refrigeration circuit, or simple ice-bath. In the event the heat exchanger is to be located in the circuit prior to the pump, a clinically available heat exchanger could be used. The sizes of the various orifices and dimensions must be capable of yielding typical flow rates out the distal end of the catheter in the range of 2-20 milliliters per minute, more preferably 3-7 milliliters per minute. Total volume delivered into the catheter would be in the range of from 40 to 200 milliliters per minute, or preferably 50-100 milliliters per minute. Expected percentage of total input delivered distally would be 2-50 percent, more preferably 10-20 percent. The typical cooling fluid temperatures would be in the range of 0-20 degrees C., preferably 5-10 degrees C. at the proximal entry point of the catheter and 15-30 degrees C., preferably 15-20 degrees C. at the distal exist point of the catheter.

In use the catheter of the present invention is transluminally introduced via an arterial insertion site such as a femoral, subclavian or brachial artery over a guide wire and through a guide catheter. The distal end of the catheter is advanced into the heart and, more specifically, into the right or left coronary artery. The proximal ends are connected to the cooling fluid handling equipment and pumping of cooling fluid is commenced. The flow rates are adjusted so as to achieve the desired cooling effect without excessive dilution of the oxygenated blood in the vascular bed. Once the desired temperature has been achieved in the myocardial tissue, the flow into the vascular bed can be reduced and maintained for as long as desired.

While the present invention has been described herein with respect to the exemplary embodiments and the best mode for practicing the invention, it will be apparent to one of ordinary skill in the art that many modifications, improvements and subcombinations of the various embodiments, adaptations and variations can be made to the invention without departing from the spirit and scope thereof. Accordingly, it is not intended that the invention be limited except by the appended claims.

What is claimed is:

1. A catheter for cooling tissue, comprising:
a supply lumen for conducting cooled fluid distally toward said tissue;
a return lumen for conducting a first portion of said fluid proximally away from said tissue; and
a distal port for delivering a second portion of said fluid to said tissue.

2. The catheter of clam 1, wherein said supply and return lumens are coaxially arranged.

3. The catheter of claim 2 wherein said supply lumen is disposed within said return lumen.

4. The catheter of claim 1, wherein said portions are adjustable.

5. The catheter of claim 4, further comprising a valve disposed near said distal end of said catheter for adjusting said portions.

6. The catheter of claim 5, wherein said valve is adjustable from the proximal end of said catheter.

7. The catheter of claim 5, wherein said valve comprises a needle valve.

8. The catheter of claim 5, wherein said valve comprises a slide valve.

9. The catheter of claim 1 wherein flow of said cooled fluid through said supply lumen and through said return lumen is generated by positive pressure applied to the fluid in the supply lumen.

10. The catheter of claim 1, wherein flow of said cooled fluid through said supply lumen is generated by positive pressure applied to the fluid in the supply lumen and wherein flow of said fluid through said return lumen is assisted by negative pressure applied to the fluid in the return lumen.

11. A percutaneously introduced catheter for cooling tissue, comprising:
a first lumen for conducting a first flow of cooled fluid toward said tissue; and
a second lumen for insulating said fluid being supplied to said tissue with a second flow of cooled fluid.

12. The catheter of claim 11, wherein said second flow comprises a portion of said first flow.

13. The catheter of claim 12, wherein said second flow is conducted away from said tissue.

14. The catheter of claim 12, wherein the difference between said first and said second flows is delivered to said tissue.

15. The catheter of claim 12 wherein said first lumen and said second lumen are coaxially arranged.

16. The catheter of claim 12, wherein said first and second flows are adjustable.

17. The catheter of claim 16, further comprising a valve for adjusting said flows.

18. The catheter of claim 17, wherein said valve is disposed near the distal end of said catheter.

19. The catheter of claim 18, wherein said valve comprises a needle valve.

20. The catheter of claim 18, wherein said valve comprises a slide valve.

21. A percutaneously introduced catheter for cooling tissue, configured for conducting a flow of cooling fluid to and from said tissue such that a portion of said flow comprising less than all of said flow is expelled from said catheter into said tissue.

22. The catheter of claim 21, wherein the portion of said flow that is expelled from said catheter is adjustable.

23. The catheter of claim 22, wherein catheter is configured such that said portion of said flow that is expelled from said catheter is adjustable while said flow of cooling fluid is being conducted toward and away from said tissue.

24. The catheter of claim 21, wherein said catheter is configured such that fluid flowing away from said tissue insulates fluid flowing to said tissue.

25. The catheter of claim 24, wherein said catheter is configured such that fluid flowing away from said tissue surrounds fluid flowing to said tissue.

26. A method for treating acute myocardial infarction in a patient comprising:
percutaneously introducing into the patient a catheter having a distal end and advancing said distal end into the aorta;
causing cooled fluid to flow distally through said catheter at a flow rate higher than is intended for delivery into a myocardial bed;
allowing only a flow rate that is intended for delivery to said myocardial bed to be expelled from near the distal end of said catheter; and
causing the remainder of said flow to flow proximally through said catheter to outside said patient.
